(19) United States
(12) Patent Application Publication
(10) Pub. No.: US 2022/0404344 A1
(43) Pub. Date: Dec. 22, 2022

(54) SILICA SHELL ENCAPSULATED POLYAROMATIC-CORE MICROPARTICLES

(71) Applicant: Arizona Board of Regents on Behalf of the University of Arizona, Tucson, AZ (US)

(72) Inventors: Craig A. Aspinwall, Tucson, AZ (US); Minhui Han, Tucson, AZ (US); Colleen Janczak, Tucson, AZ (US)

(21) Appl. No.: 17/777,011

(22) PCT Filed: Nov. 15, 2020
(86) PCT No.: PCT/US2020/060638

§ 371 Date: May 13, 2022

Related U.S. Application Data
(60) Provisional application No. 62/936,282, filed on Nov. 15, 2019.

(51) Int. Cl. G01N 33/547; G01N 33/533

(52) U.S. Cl. CPC G01N 33/547; G01N 33/533

(57) ABSTRACT

The present invention provides silica shell encapsulated polyaromatic-core microparticles and methods for producing and using the same. In particular, the silica shell encapsulated polyaromatic-core microparticles of the invention are hydrophilic microparticle scintillators comprising (i) polyaromatic-core microparticle (1), wherein said polyaromatic-core microparticle (1) is doped with a scintillator material (2); and (ii) a silica-shell portion (3) encapsulating said polyaromatic-core microparticle (1), wherein said silica-shell portion (3) comprises an outer surface (4). The polyaromatic-core portion is formed from an aromatic vinyl compound selected from the group consisting of styrene, vinyl toluene, and a mixture thereof.

### CROSS-REFERENCE TO RELATED APPLICATIONS

This application claims the priority benefit of U.S. Provisional Application No. 62/936,282, filed Nov. 15, 2019, which is incorporated herein by reference in its entirety.

### STATEMENT REGARDING FEDERALLY FUNDED RESEARCH

This invention was made with government support under Grant No. 1807343 awarded by NSF. The government has certain rights in the invention.

### FIELD OF THE INVENTION

The present invention relates to a silica shell encapsulated polystyrene-core microparticles and methods for producing and using the same.

### BACKGROUND OF THE INVENTION

Commercially available scintillating microparticles consist of inorganic crystals, or polymer (e.g., polystyrene or polyvinyltoluene) particles. There are many disadvantages to these commercially available scintillating microparticles. For example, the particle size and/or the shape of inorganic crystal based scintillating microparticles are often not uniform, thereby possibly leading to different quantitative results from one batch-to-another or requiring standardization for each experiment. Furthermore, polymer based commercially available scintillating microparticles are hydrophobic, which can lead to aggregation, and often are of low density, which causes the microparticles to float, thereby making it difficult to use in aqueous medium. Moreover, while it is possible to functionalize the surface of commercially available inorganic crystal or polymer based scintillating microparticles for different assays or purposes, it is rather time consuming and limited in scope.

Therefore, there is a need for a scintillating microparticle that is hydrophilic, relatively uniform in size, and/or where the surface modification can be readily achieved and offers a diverse surface functionalization.

### SUMMARY OF THE INVENTION

The invention provides a polymer-based hydrophilic microparticle and methods for producing and using the same. For example, hydrophilic microparticles of the invention can be used in conjunction with a scintillator such that the microparticles can be used in a scintillation proximity assay (“SPA”), or other radioassays.

One particular aspect of the invention provides a hydrophilic microparticle scintillator comprising:
a polyaromatic-core microparticle (1), wherein said polyaromatic-core microparticle (1) is doped with a scintillator material (2); and
a silica-shell portion (3) encapsulating said polyaromatic-core microparticle (1), wherein said silica-shell portion (3) comprises an outer surface (4).
   The hydrophilic microparticle described above is schematically illustrated in FIG. 1. As can be seen, the polyaromatic-core microparticle (1) is encapsulated within a silica-shell portion (3) having a thickness of d₁. In some embodiments, the silica-shell thickness d₁ ranges from about 1 nm to about 1,000 nm. Alternatively, the thickness (d₁) of the silica-shell portion (3) is no more than about 1000 nm. Yet in other embodiments, the polyaromatic-core microparticle (1) is doped with a scintillator material (2). In this manner, the hydrophilic microparticle of the invention can be used in aqueous medium SPA and other radioassays. Polyaromatic-core microparticle (1) can be polystyrene-core microparticle, polyvinyltoluene-core, or a mixture of polystyrene and polyvinyltoluene core microparticle. In some embodiments, the polyaromatic-core microparticle (1) comprises a plurality of scintillator materials (2). In one particular embodiment, each of the polyaromatic-core microparticle (1) comprises at least about 10⁵ molecules, typically at least about 10⁶ molecules, and often at least about 10⁸ molecules of scintillator materials (2). In another embodiment, each of the polyaromatic-core microparticle (1) comprises at least from about 10⁵ to about 10¹² molecules, typically at least from about 10⁶ to about 10¹⁰ molecules, and often at least from about 10⁶ to about 10⁸ molecules of scintillator materials (2). Still in another embodiment, each of the polyaromatic-core microparticle (1) comprises at least from about 10⁻¹⁹ to about 10⁻¹⁰ moles, typically at least from about 10⁻¹⁸ to about 10⁻¹¹ moles, and often at least from about 10⁻¹⁶ to about 10⁻¹² moles of scintillator materials (2).

In some embodiments, the outer surface (4) of the silica-shell portion (3) further comprises a functional group that is adapted for attaching a probe moiety. Exemplary functional groups that can be present on the outer surface (4) include, but are not limited to, a hydroxy group, an amine group, a thiol group, a chloro-silane group, a carboxylate group, an ester, an imide group, an isothiocyanate group, a halide, and other functional groups known to one skilled in the art. In this manner, a wide variety of probe moieties can be attached to the outer surface (4).

Exemplary probes that can be attached to the outer surface (4) include, but are not limited to, an oligonucleotide, an antibody, an antigen, a hormone, a receptor, an aptamer, a peptide, a chelator, a metal ion, an enzyme, a natural or synthetic receptor, and other probes known to one skilled in the art.

It should be appreciated that the probe can be attached directly to the outer surface (4) or it can be attached via a linker. When a linker is present, typically the linker is a polymer, often a hydrophilic polymer. Exemplary polymers that can be used as a linker include, but are not limited to, polyethylene glycol (PEG), polyethylenimine (PEI), polyvinyl alcohol (PVA), polyethylene oxide, polyacrylamide, agarose, and a combination thereof, as well as other polymers and combinations of polymers that are known and used as a linker in various applications by one skilled in the art.

Exemplary scintillator materials that can be used in the invention include, but are not limited to, p-terphenyl (PTP); 1,4-bis (4-methyl-5-phenyl-2-oxazolyl)benzene (dimethyl POPOP), and a mixture thereof, as well as other scintillator materials that are known to one skilled in the art.

Other aspects of the invention provide methods for producing and using the hydrophilic microparticles. For example, hydrophilic microparticles comprising scintillator can be used in scintillation proximity assay for a separation-free, selective detection of radiolabeled ligands (i.e., analytes).

Another particular aspect of the invention provides a method for producing a hydrophilic microparticle scintillator. The method generally includes:
producing a polyaromatic-core microparticle (1) that is doped with a scintillator material (2); and
contacting the polyaromatic-core microparticle (1) with a silica-shell precursor mixture under conditions sufficient to encapsulate the polyaromatic-core microparticle (1) with a silica-shell portion (3) to produce the hydrophilic microparticle scintillator.
   The hydrophilic microparticle scintillator produced comprises: a polyaromatic-core microparticle (1), wherein the polyaromatic-core microparticle (1) is doped with at least one scintillator material (2); and a silica-shell portion (3) encapsulating said polyaromatic-core microparticle (1).

The silica-shell portion (3) can be prepared by using a silica-shell precursor mixture. Typically, the silica shell precursor mixture comprises a silica shell forming reagent and a silica shell surface functional group forming agent. However, it should be appreciated that use of the silica shell surface functional group forming agent can be optional. Exemplary silica-shell forming agent that can be used to produce the silica-shell portion (3) include, but are not limited to, tetra(C₁-C₁₂)alkylorthosilicates, such as tetraethylorthosilicate (TEOS), as well as others known to one skilled in the art. Exemplary silica shell surface functional group forming agent include, but are not limited to, (3-aminopropyl)triethoxysilane (APTES), (3-mercaptopropyl)trimethoxysilane (MPTS), a mixture thereof, and others known to one skilled in the art.

In some embodiments, the step of producing polyaromatic-core microparticle (1) comprises:
polymerizing an aromatic compound (e.g., styrene or vinyl toluene) under conditions sufficient to produce a polyaromatic-core microparticle; and
admixing said polyaromatic microparticle with said scintillator material (2) under conditions sufficient to produce said polyaromatic-core microparticle (1), wherein said polyaromatic-core microparticle (1) is doped with at least one scintillator material (2).

Still in other embodiments, the step of producing the polyaromatic microparticle comprises providing a solution of an aromatic compound (i.e., styrene or vinyltoluene, or a combination thereof) and a radical polymer initiator under conditions sufficient to produce the polystyrene microparticle. Yet in other embodiments, the solution further comprises a disintegrant or an emulsifier.

Still another aspect of the invention provides a method for detecting the presence of a target ligand in a sample, said method comprising:
(i) contacting a hydrophilic microparticle scintillator described herein, in which a probe is attached to the outer surface (4) with a fluid sample under conditions sufficient to form a probe-ligand complex when the target ligand is present in the fluid sample; and
(ii) determining the formation or presence of the probe-ligand complex, thereby detecting the presence of the target ligand in the sample.

In some embodiments, the method of determining the formation or presence of said probe-ligand complex comprises determining emission of photons resulting from an interaction between radioactive decay of a radioisotope present in the target ligand and the scintillator material within the microparticle. In some instances, the radioactive decay produces α-particles, β-particles, or a combination thereof.

Still in other embodiments, the probe moiety comprises an oligonucleotide, an antibody, an antigen, a hormone, a receptor, an aptamer, a peptide, a chelator, a metal ion, an enzyme, or a natural or synthetic receptor.

Many of the reagents, starting materials, procedures, as well as other aspects of the invention can be gleamed from a commonly assigned U.S. provisional patent application No. 62/803,448, filed Feb. 9, 2019, which is incorporated herein by reference in its entirety, along with any cited references therein.

### BRIEF DESCRIPTION OF THE DRAWINGS

FIG. 1 is a schematic illustration of one embodiment of a hydrophilic microparticle scintillator of the invention.

FIG. 2 shows measured zeta potentials of scintillator doped polystyrene-core microparticles (black), hydrophilic microparticles (red) and amine functionalized hydrophilic microparticles (yellow) suspended in 100 mM NaCl aqueous solution as a function of pH.

FIG. 3 shows solvent effect on doping scintillators in polystyrene-core microparticles in terms of scintillation response to ³H. Polystyrene-core was incubated with 5×10⁻³ M scintillator doping solution for 1 h.

FIG. 4 is a graph showing the results of scintillation proximity assay for biotin/NeutrAvidin model based on the hydrophilic microparticle of the invention. ³H-labeled biotin responsive scintillant microparticles response to ³H-labeled biotin (red circle), ³H-labeled sodium acetate (blue square); silica-shell polystyrene-core scintillant microparticles response to ³H-labeled biotin (black triangle).

FIG. 5 shows a graph of scintillation response of ³H-labeled NeutrAvidin responsive scintillant microparticles (not passivated with PEG) to ³H-labeled NeutrAvidin (blue circle) and ³H-sodium acetate (black cross).

FIG. 6 is a graph showing scintillation response of ³H-labeled neutravidin responsive scintillant microparticles passivated with PEG (circle) or non-passivated with PEG (square) to ³H-labeled NeutrAvidin (red) and ³H-labeled sodium acetate (blue).

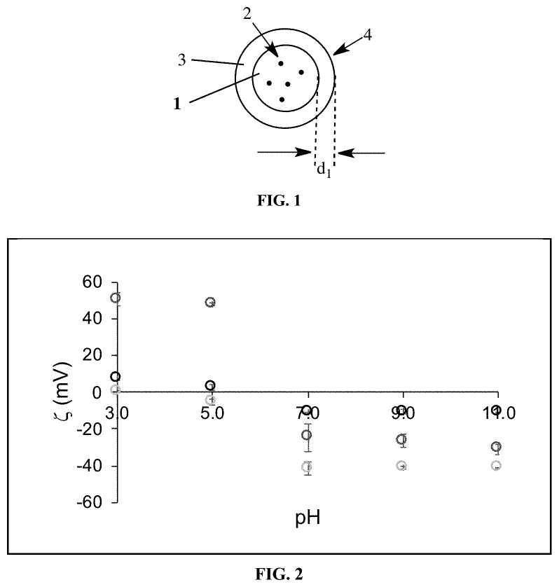

### DETAILED DESCRIPTION OF THE INVENTION

Radionuclides, such as ³H, ¹⁴C, ³²P, ³³P, and ³⁵S, are commonly used as bioanalytical labels and tracers in a wide range of biological, chemical and environmental assays due to the prevalence of H, C, P and S. However, these radioisotopes are challenging to quantify due to their low decay energies and short β-particle penetration depths in aqueous media. Alpha-particle emitting radionuclides are further used in energy and medical applications. Many alpha-particle radionuclides suffer from similar difficulties in detection due to short penetration depths in aqueous samples. Unlike fluorescent or fluorogenic probes and fluorescent protein tags, radionuclides do not significantly increase the size or mass of the labeled component, and therefore have minimal effects on binding, conformational changes, diffusion and active transport, etc. In addition, lower background signals are typically obtained for radioassays compared to fluorescence assays. In general, radioassays are among the most sensitive analytical measurements

At least partly because of these advantages radiolabeling techniques have been used to in a wide variety of assays, e.g., to investigate biochemical pathways, enzyme activity and ligand-receptor binding events. More recently, radionuclide derived pharmaceuticals have found renewed interest for treatment of cancer. Radionuclides are also used in vivo imaging in clinical settings. Overall, radioassays provide high sensitivity compared to fluorescence assays, thus enabling quantification at sub-pM concentrations.

Commercially available polymer based scintillating microparticles are not well suited for use in aqueous media due at least in part to their hydrophobic properties and/or low density, thereby often leading to aggregation and/or floatation in an aqueous medium. As for commercially available inorganic crystal based scintillating microparticles, they suffer from being denser (compared to polymer based scintillating microparticles) and having irregular shape, thereby often leading to quick settling in aqueous media and inconsistent probe-ligand complex formation.

Hydrophilic microparticle scintillators of the invention overcomes many of these shortcomings of currently commercially available scintillating microparticles. In particular, the hydrophilic microparticle scintillator of the invention comprises a polystyrene-core microparticle that is encapsulated with a silica-shell portion.

Without being bound by any theory, it is believed that the presence of a silica shell increases solubility and dispersability of the polystyrene core microparticle in aqueous medium. As used herein, the term “hydrophilic” when referring to hydrophilic microparticles of the invention means hydrophilic microparticles does not form aggregate when about 1 g, typically about 10 g, often about 20 g, more often about 30 g, still more often about 50 g, yet more often about 75 g, and most often about 100 g of hydrophilic microparticles are admixed with 1 L of water. Alternatively, the term “hydrophilic” when referring to hydrophilic microparticles of the invention means hydrophilic microparticles are closed packed when there are more particles than water per given volume.

The polyaromatic-core microparticle (1) has mean particle size in the range of from about 1 μm to about 1000 μm, typically from about 1 μm to about 500 μm, often from about 1 μm to about 100 μm, more often from about 1 μm to about 50 μm, still more often from about 1 μm to about 10 μm, and most often from about 1 μm to about 5 μm. Throughout this disclosure, the term “about” refers to being within an acceptable error range for the particular value as determined by one of ordinary skill in the art, which will depend in part on how the value is measured or determined, i.e., the limitations of the measurement system, i.e., the degree of precision required for a particular purpose. For example, the term “about” can mean within 1 or more than 1, often within 1, standard deviation, per the practice in the art. Alternatively, the term “about” when referring to a numerical value can mean±20%, typically ±10%, often ±5% and more often ±1% of the numerical value. In general, however, where particular values are described in the application and claims, unless otherwise stated, the term “about” means within an acceptable error range for the particular value.

In another embodiment, D₉₀ particle size of the polystyrene-core microparticle (1) is about 10 μm, typically about 5 μm, and often 2 μm.

Yet in other embodiments, the thickness (d₁) of said silica-shell portion (3) ranges from about 1 nm to about 1,000 nm, typically from about 5 nm to about 500 nm, and often from about 10 nm to about 300 nm. Alternatively, the thickness (d₁) of the silica-shell portion (3) is no more than about 1000 nm, typically no more than about 500 nm, and often no more than 250 nm.

The hydrophilic microparticle scintillator can also include a functional group on the surface to allow attachment of a desired probe moiety. Alternatively, the probe moiety can be already attached to the hydrophilic microparticle scintillator. In some embodiments, the probe moiety is attached to the surface of the hydrophilic microparticle scintillator via a linker such as a polymer.

General procedure for producing hydrophilic microparticle scintillators of the invention can be found in commonly assigned U.S. Provisional Patent Application No. 62/803,448 (“the '448 patent application”), which has already been incorporated by reference in its entirety. It should be appreciated that one of the significant changes compared to the method disclosed in the '448 patent application is use of styrene to produce a polystyrene microparticle without having a silica shell.

For attaching a linker or a polymer to the outer surface (4) of the silica-shell portion (3) a wide variety of methods can be used. For example, after the silica-shell portion (3) is made, the functional group that is present on the surface of the silica-shell portion (3) can be used to attach the linker. Alternatively, a first silica shell layer (not shown) is made. This first silica shell layer encapsulates the polyaromatic-core microparticle (1). To this first silica shell layer, another layer of silica shell can be added that incorporates the linker. For example, using a mixture of tetraethylorthosilicate (TEOS) and silane containing polymer (e.g., polyethylene glycol-triethoxysilane (PEG-O—Si(OEt)₃), one can form a second or the outer layer of the silica-shell portion (3). In this manner, the polymer becomes an integral part of or embedded within the silica-shell portion (3).

In some embodiments, the hydrophilic microparticle scintillator includes a surface functional group that is present on the outer surface (4) of the hydrophilic microparticle scintillator. This allows attachment of a wide variety of probe moieties, thereby allowing one to tailor the hydrophilic microparticle scintillator to a desired SPA. This functional group can also be used to attach various other moieties, such as a polymer, to the hydrophilic microparticle scintillator. For example, hydrophilic polymers can be attached to the outer surface (4) to further increase hydrophilicity or to serve as a linker for attaching a probe. Exemplary polymers that are useful in increasing hydrophilicity or for attaching a probe include, but are not limited to, polyethylene glycol (PEG), polyethylenimine (PEI), polyacrylamide, agarose, polyvinyl alcohol, polyethylene oxide, as well as a combination thereof, and other hydrophilic organic polymers known to one skilled in the art.

By using the hydrophilic microparticle scintillator of the invention, a wide variety of ligands can be analyzed in an aqueous sample. It should be appreciated that unless the context requires otherwise, the terms “ligand” and “probe” do not refer to any particular substance or size relationship. These terms are only operational terms that indicate selective binding between the ligand and the corresponding probe where the moiety that is bound to the silica shell nanocomposite material is referred to as a probe and any substance that selectively binds to the probe is referred to as a ligand. Thus, if an antibody is attached to the hydrophilic microparticle scintillator then the antibody is a probe and the corresponding antigen is a ligand. However, if an antigen is attached to the hydrophilic microparticle scintillator then the antigen is a probe and the corresponding antibody is a ligand.

Polystyrene (PS) core has many advantages including, but not limited to, low cost and compatibility with a wide range of scintillant fluorophores. The microparticle scintillators of the invention are hydrophilic and as such do not require addition of surfactants when used in an aqueous media. In contrast, commercially available microparticle scintillators may require addition of surfactants when used in aqueous media. In some embodiments, by using polyaromatic-core microparticle (1), the density of the hydrophilic microparticle scintillators of the invention remain dispersed in an aqueous media without forming aggregate. The hydrophilic microparticle scintillators of the invention can be easily recovered, e.g., via centrifugation, enabling sample enrichment and isolation as well as reuse of the hydrophilic microparticle scintillator if desired.

The hydrophilic microparticle scintillators of the invention can be used in a wide variety of apparatuses and applications. For example, hydrophilic microparticle scintillators of the invention can be used in scintillation proximity assay, for particle-packed cartridges, detection cuvettes, separations columns, as well as for pull-down assays and assays in which lipid coatings or lipid-stabilized membrane proteins are incorporated. By using hydrophilic scintillator microparticles in place of traditional silica or polymer microparticles in these applications, the superior sensitivity of radioisotope detection could be coupled to established separations techniques. For example, a scintillant microparticle packed detection cuvette could be used in the on-line detection of radioisotope labeled analytes during liquid chromatography separations. In another example, a scintillant microparticle-packed cartridge could be used in the testing of wastewater or effluent in which there is some possibility of contamination by radioactive materials, where the sample is collected in the field, but the measurement is made later, in a laboratory.

One method of determining the hydrophilicity of hydrophilic microparticle scintillators of the invention is to measure the surface charge or the zeta potential. In some embodiments, the zeta potential as determined using the procedure described in the Examples section, of hydrophilic microparticle scintillators of the invention is about −20 mV or less, typically about −25 mV or less, and often about −30 mV or less at pH 7. Still in other embodiments, the zeta potential of hydrophilic microparticle scintillators of the invention is about −25 mV or less, typically about −30 mV or less, and often about −40 mV or less at pH 9. Yet in other embodiments, the zeta potential of hydrophilic microparticle scintillators of the invention is about −30 mV or less, typically about −35 mV or less, and often about −40 mV or less at pH 11. In one particular embodiment, the zeta potential of hydrophilic microparticle scintillators of the invention is about 30 mV or more, typically about 40 mV or more, and often about 50 mV or more at pH 3. In another particular embodiment, the zeta potential of hydrophilic microparticle scintillators of the invention is about 30 mV or more, typically about 40 mV or more, and often about 45 mV or more at pH 5.

Another advantage of hydrophilic microparticle scintillators of the invention is that unlike hydrophilic scintillators that float and/or form aggregates in an aqueous medium, the hydrophilic microparticle scintillators of the invention do not float or form large aggregates in an aqueous medium. In particular, the density of the hydrophilic microparticle scintillators of the invention is such that they do not float on top of an aqueous medium or settle rapidly. In one particular embodiment, the density of the hydrophilic microparticle scintillators of the invention is at least about 1.1 g/mL, typically about at least 1.15 g/mL, and often at least about 1.2 g/mL. Alternatively, the density of the hydrophilic microparticle scintillators of the invention ranges from about 1.1 g/mL to about 1.4 g/mL, typically from about 1.1 g/mL to about 1.3 g/mL, and often from about 1.1 g/mL to about 1.2 g/mL.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Preparation of polystyrene-core microparticles: To a 50 ml round bottle flask with magnetic stir bar, 20 g of methanol was first poured into the vial and 2 g styrene, 10 wt % relative to the medium, was charged. The amount of VA-057 (FUJIFILM Wako Pure Chemical, Tokyo, JAPAN), i.e., 2,2′-Azobis[N-(2-carboxyethyl)-2-methylpropionamidine]tetrahydrate—a radical polymer initiator, was kept at 2 wt % (0.04 g) and the concentrations of polyvinylpyrrolidone (i.e., PVP, an emulsifier and disintegrant for solution polymerization) was kept at 2 wt % based on styrene. The polymerization temperature was fixed at 60° C. in oil bath overnight. After completion of the polymerization, the result was rinsed off with nanopure water and methanol, then centrifuged repeatedly to remove the residual styrene and PVP. The polystyrene-core microparticles were eventually stored in nanopure water for future usage.

It should be appreciated that other radical polymerization initiators such as AIBN and other azo initiators (see, for example, wako-chem.co.jp/kaseihin_en/waterazo/index.htm) can be used instead of VA-057. Furthermore, instead of or in combination with PVP, other emulsifier and disintegrant for solution polymerization known to one skilled in the art can be used.

Preparation of scintillator-doped polystyrene-core microparticles: 120 mg of purified polystyrene-core microparticles were suspended in 6 mL of nanopure water (2 wt %). To suspension, 1 mL of scintillator solution (in THF/H₂O mixture or CHCl₃/isopropanol mixture) was added and vortexed for homogeneous mixing. The mixture was incubated for 1 h, then centrifuged repeatedly to remove the supernatant. The scintillator-doped polystyrene-core microparticles were eventually stored in water for future usage.

Preparation of silica-shell polystyrene-core scintillant microparticles: A mixture of scintillant microparticles (50 mg, solid content: 7.5 wt % in suspension), 6.67 g of ethanol and 0.267 g of NH₄OH were charged into a 50 ml centrifuge tube. Then, 0.67 g of silane was injected into the mixture suspension. The mixture was incubated overnight to allow the formation of silica shell on particles.

Preparation of ³H-labeled biotin responsive scintillant microparticles NeutrAvidin was dissolved in water at a concentration of 2 mg/mL for the stock solution. 250 μL of NeutrAvidin stock solution was incubated with 250 μL of PBS buffer (10 mM, pH 7.4). To 3 mg of silica-shell polystyrene-core scintillant microparticles, 500 μL of NeutrAvidin solution was added and incubated overnight. Excess NeutrAvidin was removed by centrifugal filtration and the purified particles was finally suspended in 1 mL of PBS buffer for further usage.

Preparation of ³H-labeled NeutrAvidin responsive scintillant microparticles: 50 mg of amine-functionalized silica-shell polystyrene-core scintillant microparticles were incubated with 5 mg of biotin-PEG-NHS (3 k Da) in 10 mL of PBS buffer (10 mM, pH 7.4) overnight. The particles were washed several times with water. To reduce nonspecific adsorption of NeutrAvidin, biotin crosslinked scintillant particles were passivated by PEG. The particles were washed and resuspended in anhydrous acetonitrile (5 mL), incubated with PEG₆₋₉ dimethyl chlorosilane (2% w/v) overnight. The passivated particles were successively washed with acetonitrile, ethanol and PBS buffer and finally suspended in PBS buffer for future usage.

Zeta Potential Measurements: Zeta potential of microparticles with varying compositions was measured using a Zetasizer Nano (Malvern Instruments, Ltd., Malvern, U.K.). Polystyrene and hydrophillic microparticles were dispersed in 100 mM NaCl at pH from 3.0 to 11.0 immediately prior to measurement. The zeta potential was calculated using the Smoluchowski approximation as the solution for the Henry equation for all samples.

Characterization of hydrophilic microparticles: Hydrophilic scintillant microparticles were prepared as described with a polystyrene core and a silica shell. Following preparation, the surface charge of different microparticle compositions was measured (FIG. 2). Briefly, the zeta potential for scintillator doped polystyrene-core microparticles (black), hydrophilic microparticles (red) and amine functionalized hydrophilic microparticles (yellow) are as follows:

black (x,y): (3,6.9); (5,2.5); (7, −10.9); (9, −10.6); and (11,−11.3);

red (x,y): (3,50.4); (5,47.9); (7,−24.7); (9,−26.7); and (11,−31.2); and

yellow (x,y): (3,0.4); (5,−5.1); (7,−41.6); (9,−41.2); and (11,−41.3).

When only polystyrene was utilized, the zeta potential was near neutral values ranging from +7 to −11 mV as a function of pH. The near neutral zeta potential leads to aggregation of polystyrene particles. In contrast, when hydrophilic microparticles were prepared by adding a silica coating, the surface charge as a function of pH was significantly different than the polystyrene microparticles. When further surface modification was performed using an amine-modifier, the pH-dependent surface charge was again different, indicative of a hydrophilic surface.

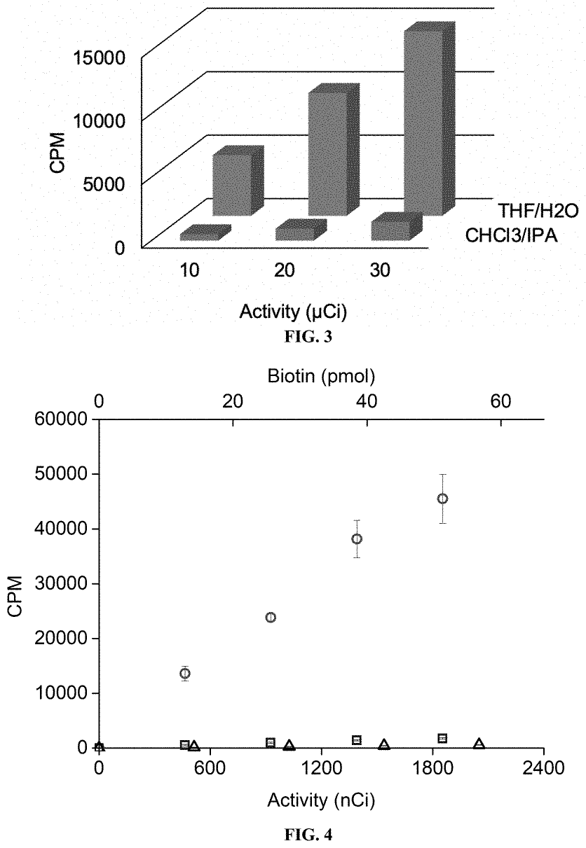

Testing of scintillant response as a function of scintillant doping conditions. To determine the optimal process for loading scintillant dyes into polystyrene microparticle cores, polystyrene cores were added to a scintillant solutions prepared using a THF/water or chloroform/isopropanol mixture. The scintillants were removed with excess washing and the polystyrene particles were suspended in water. ³H labeled sodium acetate was added and the scintillation response was measured. As seen in FIG. 3, significantly higher scintillation responses were observed in polystyrene microparticles prepared using the THF/water mixture to prepare the scintillant core.

Testing of biotin responsive scintillant microparticles in a model scintillation proximity assay: Biotin responsive scintillant microparticles were dispersed at 3 mg/mL in PBS in 7 mL plastic scintillation vials and then treated with increasing activities of ³H-labeled biotin immediately before the measurement of scintillation response in a liquid scintillation counter. For comparison, separate samples of biotin responsive scintillant microparticles were also treated with ³H-labeled sodium acetate, which is not expected to bind to the NeutrAvidin on the microparticle surface. In addition, to observe the level of non-specific binding of biotin to the scintillant microparticles, samples of scintillant microparticles lacking NeutrAvidin were treated with ³H-biotin. The response of the scintillant microparticles to ³H-biotin or ³H-sodium acetate can be seen in the plot shown in FIG. 4.

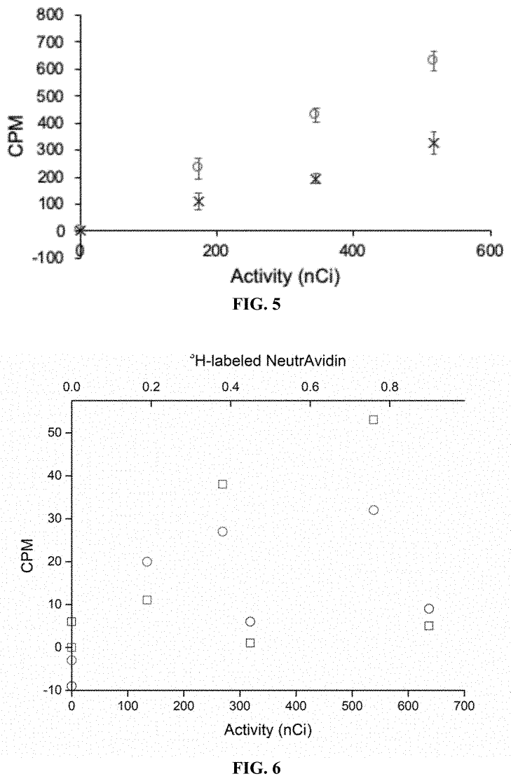

Comparison of scintillant microparticles with PEG-modified surfaces to scintillant microparticles without PEG: Scintillant microparticles modified with PEG₆₋₉ dimethyl chlorosilane as described above were dispersed at 3 mg/mL in PBS and treated with increasing activities of ³H-labeled NeutrAvidin immediately before the measurement of scintillation response with a liquid scintillation counter. Scintillant microparticles that were not modified at the silica surface were also treated with increasing activities of ³H-NeutrAvidin for comparison. The scintillation response of PEG₆₋₉ dimethyl chlorosilane modified and unmodified scintillant microparticles can be seen in the plot shown in FIG. 5. The reduction in non-specific adsorption of ³H-labeled NeutrAvidin is approximately 3× at the activities tested.

Testing of NeutrAvidin responsive scintillant microparticles in a model scintillation proximity assay: Scintillant microparticles functionalized with biotin and with PEG₆. 9 dimethyl chlorosilane were dispersed at 3 mg/mL in PBS and treated with increasing activities of ³H-labeled NeutrAvidin or ³H-labeled sodium acetate for 24 hours before measurement of scintillation response with a liquid scintillation counter. See FIG. 6. Scintillant microparticles not modified using PEG₆₋₉ dimethyl chlorosilane were also treated with increasing activities of ³H-labeled NeutrAvidin or ³H-labeled sodium acetate for comparison. See FIG. 6. The passivation of the silica shell surfaces of the scintillant microparticles by PEG appears to reduce non-specific adsorption of the ³H-labeled NeutrAvidin.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1. A hydrophilic microparticle scintillator comprising:
a polyaromatic-core microparticle (1), wherein said polyaromatic-core microparticle (1) is doped with a scintillator material (2); and
a silica-shell portion (3) encapsulating said polyaromatic-core microparticle (1), wherein said silica-shell portion (3) comprises an outer surface (4).

2. The hydrophilic microparticle scintillator of claim 1, wherein the outer surface (4) of said silica-shell portion (3) further comprises a functional group that is adapted for attaching a probe moiety.

3. The hydrophilic microparticle scintillator of claim 2, wherein said functional group is selected from the group consisting of a hydroxy group, an amine group, a thiol group, a chloro-silane group, a carboxylate group, an ester, an imide group, an isothiocyanate group, and a halide.

4. The hydrophilic microparticle scintillator of claim 1, wherein the outer surface (4) of said silica-shell portion (3) further comprises a probe moiety that is capable of selectively binding to a ligand molecule, wherein said probe moiety is attached to the outer surface (4) of said silica-shell portion (3) optionally via a linker.

5. The hydrophilic microparticle scintillator of claim 4, wherein said probe moiety is attached to the outer surface (4) of said silica-shell portion (3) via a linker.

6. The hydrophilic microparticle scintillator of claim 5, wherein said linker comprises a polymer that is attached to the outer surface (4) of said silica shell portion (3).

7. The hydrophilic microparticle scintillator of claim 6, wherein said polymer is a hydrophilic polymer.

8. The hydrophilic microparticle scintillator of claim 7, wherein said polymer comprises polyethylene glycol (PEG), polyethylenimine (PEI), polyvinyl alcohol (PVA), polyethylene oxide, polyacrylamide, agarose, or a combination thereof.

9. The hydrophilic microparticle scintillator of claim 4, wherein said probe moiety comprises an oligonucleotide, an antibody, an antigen, a hormone, a receptor, an aptamer, a peptide, a chelator, a metal ion, an enzyme, or a natural or synthetic receptor.

10. The hydrophilic microparticle scintillator of claim 1, wherein said polystyrene-core microparticle (1) has mean particle size in the range of from about 1 μm to about 1000 μm.

11. The hydrophilic microparticle scintillator of claim 1, wherein D₉₀ particle size of said polystyrene-core microparticle (1) is about 2 μm.

12. The hydrophilic microparticle scintillator of claim 1, wherein the average particle size of said polystyrene-core microparticle (1) ranges from about 1 μm to about 5 μm.

13. The hydrophilic microparticle scintillator of claim 1, wherein a thickness (d₁) of said silica-shell portion (3) ranges from about 10 nm to about 300 nm.

14. The hydrophilic microparticle scintillator of claim 1, wherein the thickness (d₁) of said silica-shell portion (3) is no more than about 500 nm.

15. The hydrophilic microparticle scintillator of claim 1, wherein said polyaromatic-core microparticle (1) is selected from the group consisting of a polystyrene-core microparticle, a polyvinyltoluene-core microparticle, and a polyaromatic-core that is a co-polymer of styrene and vinyltoluene.

16. The hydrophilic microparticle scintillator of claim 1, wherein a density of said hydrophilic microparticle scintillator is at least about 1.1 g/mL.

17. A method for producing a hydrophilic microparticle scintillator wherein said hydrophilic microparticle scintillator comprises:
a polyaromatic-core microparticle (1), wherein said polyaromatic-core microparticle (1) is doped with at least one scintillator material (2); and
a silica-shell portion (3) encapsulating said polyaromatic-core microparticle (1),
said method comprising:
producing said polyaromatic-core microparticle (1) that is doped with a scintillator material (2); and
contacting the polyaromatic-core microparticle (1) with a silica-shell precursor mixture under conditions sufficient to encapsulate the polyaromatic-core microparticle (1) with a silica-shell portion (3) to produce the hydrophilic microparticle scintillator,
wherein said polyaromatic-core microparticle (1) is selected from the group consisting of polystyrene-core microparticle, polyvinyltoluene-core microparticle, and a polyaromatic-core that is a co-polymer of styrene and vinyltoluene.

18. The method of claim 17, wherein said step of producing the polyaromatic-core microparticle (1) comprises:
polymerizing an aromatic vinyl compound under conditions sufficient to produce a polyaromatic microparticle, wherein said aromatic vinyl compound is selected from the group consisting of styrene, vinyl toluene, and a mixture thereof; and
admixing said polyaromatic microparticle with said scintillator material (2) under conditions sufficient to produce said polyaromatic-core microparticle (1), wherein said polyaromatic-core microparticle (1) is doped with at least one scintillator material (2).

19. The method of claim 18, wherein said step of producing the polyaromatic microparticle comprises providing a solution of an aromatic vinyl compound and a radical polymer initiator under conditions sufficient to produce the polyaromatic microparticle, wherein said aromatic vinyl compound is selected from the group consisting of styrene, vinyl toluene, and a mixture thereof.

20. The method of claim 19, wherein said solution further comprises a disintegrant and/or an emulsifier.

21. An apparatus comprising a hydrophilic microparticle scintillator comprising:
a polyaromatic-core microparticle (1), wherein said polyaromatic-core microparticle (1) is doped with a scintillator material (2); and
a silica-shell portion (3) encapsulating said polyaromatic-core microparticle (1), wherein said silica-shell portion (3) comprises an outer surface (4).

22. The apparatus according to claim 21, wherein said apparatus is a particle-packed cartridge, a detection cuvette, or a separation column.
